(19) United States
(12) Patent Application Publication
(10) Pub. No.: US 2019/0150898 A1
(43) Pub. Date: May 23, 2019

(54) ENERGY CONTROL DEVICE AND TREATMENT SYSTEM

(71) Applicant: OLYMPUS CORPORATION, Tokyo (JP)

(72) Inventors: Minoru KAWASAKI, Tokyo (JP); Yoshihiro TSUBUKU, Fuchu-shi (JP); Gen KATO, Hachioji-shi (JP)

(73) Assignee: OLYMPUS CORPORATION, Tokyo (JP)

(21) Appl. No.: 16/251,638

(22) Filed: Jan. 18, 2019

Related U.S. Application Data
(63) Continuation of application No. PCT/JP2016/071239, filed on Jul. 20, 2016.

(51) Int. Cl. A61B 8/00; A61B 17/32; A61B 8/08

(52) U.S. Cl. CPC A61B 8/56; A61B 8/5207; A61B 8/4494; A61B 17/320068

(57) ABSTRACT

An energy control device supplies power to an ultrasonic treatment instrument including an end effector performing treatment using ultrasonic vibration generated by an ultrasonic transducer. The energy control device includes an energy source configured to output the power to the ultrasonic transducer, and at least one integrated circuit. The integrated circuit is configured to: acquire a resonance frequency of a vibration system including the ultrasonic transducer; calculate an integrated value of a difference between the resonance frequency and a predetermined frequency after the acquired resonance frequency has become a first threshold; and perform causing the energy source to stop or reduce an output of the power to the ultrasonic transducer, or notifying that the integrated value has reached the second threshold when the integrated value has reached a second threshold.

### CROSS-REFERENCE TO RELATED APPLICATIONS

This application is a Continuation Application of PCT Application No. PCT/JP2016/071239, filed Jul. 20, 2016, the entire contents of which are incorporated herein by reference.

### BACKGROUND OF THE INVENTION

### 1. Field of the Invention

The present invention relates to an energy control device for an ultrasonic treatment instrument and a treatment system including the same.

### 2. Description of the Related Art

There is known an ultrasonic treatment instrument that grasps a living tissue to be treated with a pair of grasping pieces and ultrasonically vibrates one of the grasping pieces to perform coagulation, incision, or the like of the grasped living tissue. An example of such an ultrasonic treatment instrument is disclosed in International Publication No. 2012/044600. A resonance frequency of a vibration system including a grasping piece that ultrasonically vibrates decreases as temperature rises. Therefore, when the resonance frequency of the vibration system is acquired, it is possible to grasp a state of treatment that is being performed to a certain extent. In the ultrasonic treatment instrument disclosed in the above literature, a control device detects a resonance frequency of the vibration system that ultrasonically vibrates, and determines that grasping piece has become sufficiently high temperature when the resonance frequency has fallen below a certain threshold, and reduces an amplitude of ultrasonic vibration.

It is not preferable to continue the vibration in the ultrasonic treatment instrument that grasps a living tissue with the pair of grasping pieces to perform the treatment of the tissue by ultrasonic vibration even after the treatment has been completed and the tissue has been divided. If the grasping piece vibrates in a state where the pair of grasping pieces is in direct contact with each other, there is a risk that the grasping piece may be damaged. A technique of apprehending the temperature of the grasping piece by detecting the resonance frequency of the vibration system as disclosed in the above literature can also be used to detect division of the tissue to be treated, for example, to stop the output after the division.

### BRIEF SUMMARY OF THE INVENTION

According to an aspect of the present invention, an energy control device supplies power to an ultrasonic treatment instrument including an end effector performing treatment using ultrasonic vibration generated by an ultrasonic transducer. The energy control device includes an energy source and at least one integrated circuit. The energy source is configured to output the power to the ultrasonic transducer. The integrated circuit is configured to: acquire a resonance frequency of a vibration system including the ultrasonic transducer; calculate an integrated value of a difference between the resonance frequency and a predetermined frequency after the acquired resonance frequency has become a first threshold; and perform at least one of causing the energy source to stop or reduce an output of the power to the ultrasonic transducer and notifying that the integrated value has reached the second threshold when the integrated value has reached a second threshold.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a view illustrating an example of an outline of a treatment system according to one embodiment;

FIG. 2A is a view illustrating a schematic cross section perpendicular to a longitudinal axis of an end effector regarding the end effector and a tissue to be treated according to one embodiment;

FIG. 2B is a view illustrating a schematic cross section perpendicular to the longitudinal axis of the end effector regarding the end effector and the tissue to be treated according to one embodiment;

FIG. 3 is a block diagram illustrating an example of a configuration relating to supply of power from an energy control device to an ultrasonic treatment instrument according to one embodiment;

FIG. 4 is a view for describing a frequency change with respect to an elapsed time and a threshold for determination; and

FIG. 5 is a flowchart schematically illustrating an example of an output control process according to one embodiment.

### DETAILED DESCRIPTION OF THE INVENTION

<Configuration of Treatment System>

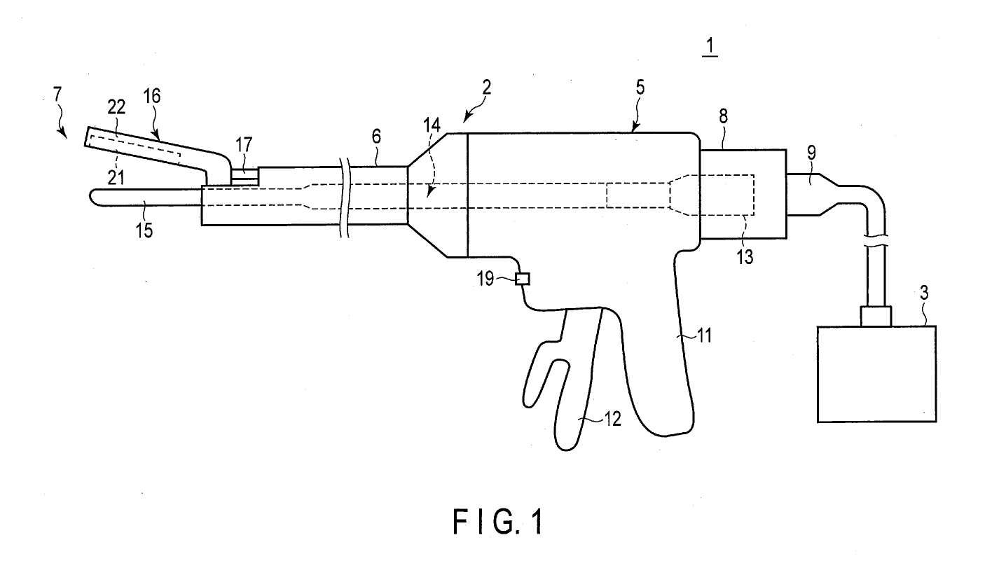

One embodiment of the present invention will be described with reference to the drawings. The present embodiment relates to a treatment system that uses ultrasonic vibration to perform treatment of a living tissue. FIG. 1 is a view illustrating a treatment system 1. As illustrated in FIG. 1, the treatment system 1 includes an ultrasonic treatment instrument 2 and an energy control device 3 that supplies power to the ultrasonic treatment instrument 2.

The ultrasonic treatment instrument 2 includes a housing 5, a shaft 6 connected to the housing 5, and an end effector 7 provided at an end of the shaft 6. A side on which the end effector 7 is provided will be referred to as a distal end side and a side on which the housing 5 is provided will be referred to as a proximal end side. The housing 5 is provided with a grip 11 and a handle 12. A user grasps the grip 11 to hold the ultrasonic treatment instrument 2. The handle 12 is configured to be opened and closed with respect to the grip 11.

An ultrasonic transducer unit 8 is provided on the proximal end side of the housing 5. The ultrasonic transducer unit 8 has an ultrasonic transducer 13 including at least one piezoelectric element. The ultrasonic transducer unit 8 is detachably connected to the energy control device 3 via a cable 9. The ultrasonic transducer 13 vibrates as AC power is supplied from the energy control device 3 to the ultrasonic transducer 13 of the ultrasonic transducer unit 8.

A rod member 14 is connected to the ultrasonic transducer 13 of the ultrasonic transducer unit 8. The rod member 14 passes through the inside of the housing 5 and the shaft 6 and reaches the end effector 7. That is, a distal end portion of the rod member 14 forms a first grasping piece 15 of the end effector 7. The rod member 14 is made of a material having a high vibration transmissibility such as a titanium alloy. The ultrasonic vibration generated by the ultrasonic transducer 13 is transmitted to the rod member 14. As a result, the first grasping piece 15 vibrates. The first grasping piece 15 vibrates at a frequency corresponding to arbitrarily designed resonance of a vibration system. This resonance frequency is, for example, about several tens of kHz although not limited thereto, and may be, for example, 46 kHz to 48 kHz (about 47 kHz).

A second grasping piece 16 is attached to a distal end portion of the shaft 6 so as to be open or closed with respect to the first grasping piece 15. The second grasping piece 16 and the handle 12 are connected by a movable member 17 passing through the inside of the shaft 6. The movable member 17 moves to the distal end side or the proximal end side by opening or closing the handle 12 with respect to the grip 11, and the second grasping piece 16 rotates with respect to the shaft 6 to be open and closed with respect to the first grasping piece 15. In this manner, the first grasping piece 15 and the second grasping piece 16 of the end effector 7 are open or closed along with the opening or closing operation of the handle 12 with respect to the grip 11. The end effector 7 is configured to grasp a living tissue to be treated by the first grasping piece 15 and the second grasping piece 16.

The second grasping piece 16 includes a pad member 21 and a holder member 22 to which the pad member 21 is attached. The pad member 21 is made of a resin such as polytetrafluoroethylene (PTFE). In a state where the first grasping piece 15 and the second grasping piece 16 are closed, the pad member 21 of the second grasping piece 16 is in contact with the first grasping piece 15, and the other portion of the second grasping piece 16 is not in contact with the first grasping piece 15.

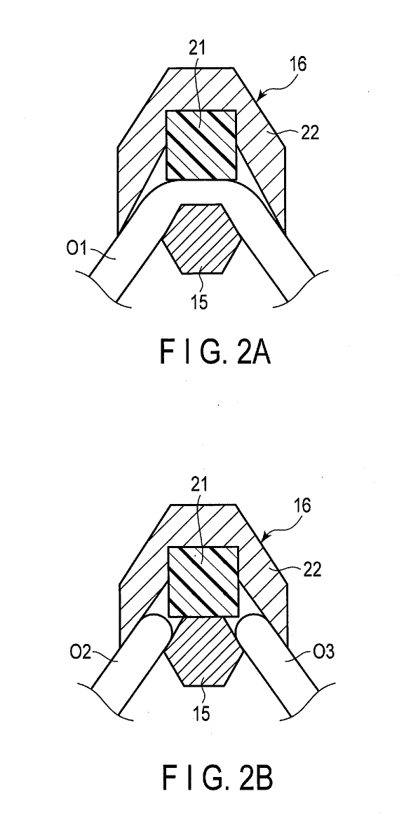

FIGS. 2A and 2B are views illustrating cross sections perpendicular to longitudinal axes of the first grasping piece 15 and the second grasping piece 16 when using the ultrasonic treatment instrument 2. FIG. 2A illustrates a state where a tissue O1 to be treated is sandwiched between the first grasping piece 15 and the second grasping piece 16. The ultrasonic treatment instrument 2 can incise the tissue O1 while coagulating the tissue O1 as the first grasping piece 15 ultrasonically vibrates with the tissue O1 sandwiched between the first grasping piece 15 and the second grasping piece 16. FIG. 2B illustrates a state where the tissue is divided into a first tissue piece O2 and a second tissue piece O3. At this time, the pad member 21 is in contact with the first grasping piece 15.

The housing 5 is provided with an operation button 19. An operation to switch on and off of the supply of power from the energy control device 3 to the ultrasonic transducer unit 8 is input to the operation button 19. Incidentally, the treatment system 1 may be provided with a foot switch having the same function as the operation button 19 instead of the operation button 19 or together with the operation button 19.

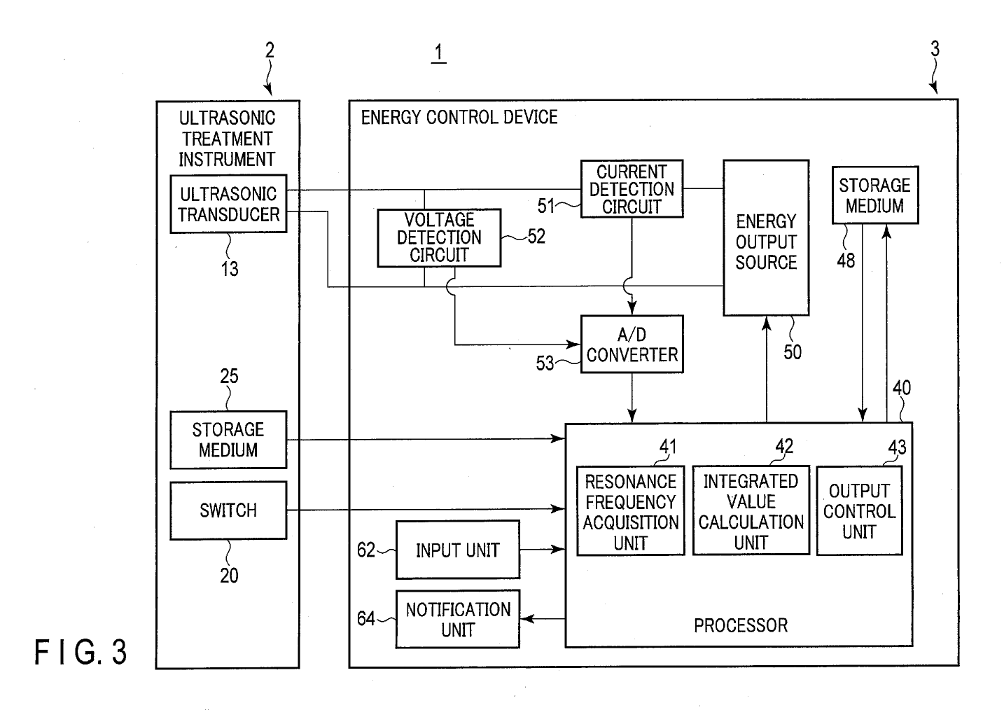

FIG. 3 is a diagram schematically illustrating a configuration example of the treatment system 1 relating to the supply of power from the energy control device 3 to the ultrasonic treatment instrument 2.

As illustrated in FIG. 3, the ultrasonic treatment instrument 2 is provided with the ultrasonic transducer 13, a switch 20, and a storage medium 25. The ultrasonic transducer 13 is an ultrasonic transducer serving as a vibration source provided in the ultrasonic transducer unit 8 described above. The switch 20 is provided inside the housing 5 of the ultrasonic treatment instrument 2. The switch 20 is switched between an on-state and an off-state as the operation is input by the operation button 19. Further, information on the treatment instrument is stored in the storage medium 25.

The energy control device 3 includes a processor 40 controlling an operation of the treatment system 1 and a storage medium 48. The processor 40 operates based on, for example, a program stored in the storage medium 48, and controls an operation of each unit of the energy control device 3. In the storage medium 48, a processing program to be used by the processor 40, a parameter, a table, and the like to be used for computation performed by the processor 40 are stored.

Further, the energy control device 3 includes an energy output source 50, a current detection circuit 51, a voltage detection circuit 52, and an A/D converter 53. The energy output source 50 supplies AC power to the ultrasonic transducer 13 of the ultrasonic treatment instrument 2. The energy output source 50 outputs power under the control of the processor 40. The current detection circuit 51 detects a current value of an output current I from the energy output source 50 to the ultrasonic transducer 13. The voltage detection circuit 52 detects a voltage value of an output voltage V from the energy output source 50 to the ultrasonic transducer 13. Analog signals output from the current detection circuit 51 and the voltage detection circuit 52 are converted into digital signals via the A/D converter 53 and the converted signals are transmitted to the processor 40.

The energy control device 3 further includes an input unit 62 and a notification unit 64. The input unit 62 is a part that receives an input of a user with respect to the energy control device 3. The input unit 62 includes, for example, a touch panel, a button switch, a keyboard, or the like. The notification unit 64 is a part that notifies the user of information. The notification unit 64 includes, for example, a display, a speaker, or the like.

The energy control device 3 according to the present embodiment performs the following operation. That is, when the switch 20 of the ultrasonic treatment instrument 2 is turned on, the supply of power to the ultrasonic treatment instrument 2 is started with detection of such an on-state. Along with the treatment, the temperature of the first grasping piece 15 of the ultrasonic treatment instrument 2 rises, and the resonance frequency of the vibration system having temperature dependence decreases. The energy control device 3 adjusts an output frequency in accordance with the changing resonance frequency. Further, a progress state of the treatment is determined based on the resonance frequency, and the output is stopped or the like. The processor 40 performs computation relating to the operation of the energy control device 3 as described above.

The processor 40 executes functions as a resonance frequency acquisition unit 41, an integrated value calculation unit 42, and an output control unit 43. The resonance frequency acquisition unit 41 acquires a resonance frequency of the vibration system including the ultrasonic transducer 13, the rod member 14, and the like of the ultrasonic treatment instrument 2. The resonance frequency acquisition unit 41 can acquire the resonance frequency based on an output current and an output voltage from the energy output source 50 supplied to the ultrasonic transducer 13. The integrated value calculation unit 42 calculates an integrated value of a difference between a resonance frequency and a first threshold after the resonance frequency of a vibration system has become the first threshold. The output control unit 43 controls the output of the energy output source 50. Such control includes detection of a state of the switch 20 and controlling of the output of the energy output source 50 based on the detection result. For example, when the integrated value of the difference between the resonance frequency and the first threshold has reached a second threshold, the output control unit 43 causes the energy output source 50 to stop or reduce the output of power to the ultrasonic transducer 13, or notifies that the integrated value has reached the second threshold.

The processor 40 includes an integrated circuit such as a central processing unit (CPU), an application specific integrated circuit (ASIC), and a field programmable gate array (FPGA). The processor 40 may be constituted by one integrated circuit or the like, or a combination of a plurality of integrated circuits or the like. Such operations of the integrated circuit are performed in accordance with programs recorded in, for example, the storage medium 48 or a recording area in the integrated circuit.

<Operation of Treatment System>

The operation of the treatment system 1 according to the present embodiment will be described. When an object to be treated such as a living tissue is treated using the treatment system 1, the user holds the grip 11 and the handle 12, and inserts the end effector 7 into a body cavity such as an abdominal cavity. The user arranges a living tissue or the like to be treated between the first grasping piece 15 and the second grasping piece 16, and closes the handle 12 with respect to the grip 11. As a result, the second grasping piece 16 is closed with respect to the first grasping piece 15, and the living tissue is grasped between the first grasping piece 15 and the second grasping piece 16.

In this state, the user turns on the operation button 19. At this time, the processor 40 detects that the switch 20 has been turned on. The output control unit 43 of the processor 40 causes the energy output source 50 to start outputting the power to the ultrasonic transducer 13. As a result, ultrasonic vibration is generated by the ultrasonic transducer 13, and the generated ultrasonic vibration is transmitted to the first grasping piece 15 via the rod member 14. When the first grasping piece 15 ultrasonically vibrates in a state where the tissue to be treated is grasped between the first grasping piece 15 and the second grasping piece 16, frictional heat is generated between the first grasping piece 15 and the grasped tissue. Due to the frictional heat, the tissue is incised at the same time of being coagulated.

As illustrated in FIG. 2B, a state where the first grasping piece 15 and the second grasping piece 16 are in contact with each other is formed in a situation where the tissue to be treated has been cut. When the first grasping piece 15 continues ultrasonic vibration in this state, the pad member 21 and the like of the second grasping piece 16 is likely to be damaged. Therefore, in the present embodiment, when the processor 40 detects the situation where the tissue has been cut, the processor 40 stops or reduce the supply of power to the ultrasonic transducer 13 from the energy output source 50, and stops or reduces the output of the ultrasonic treatment instrument 2.

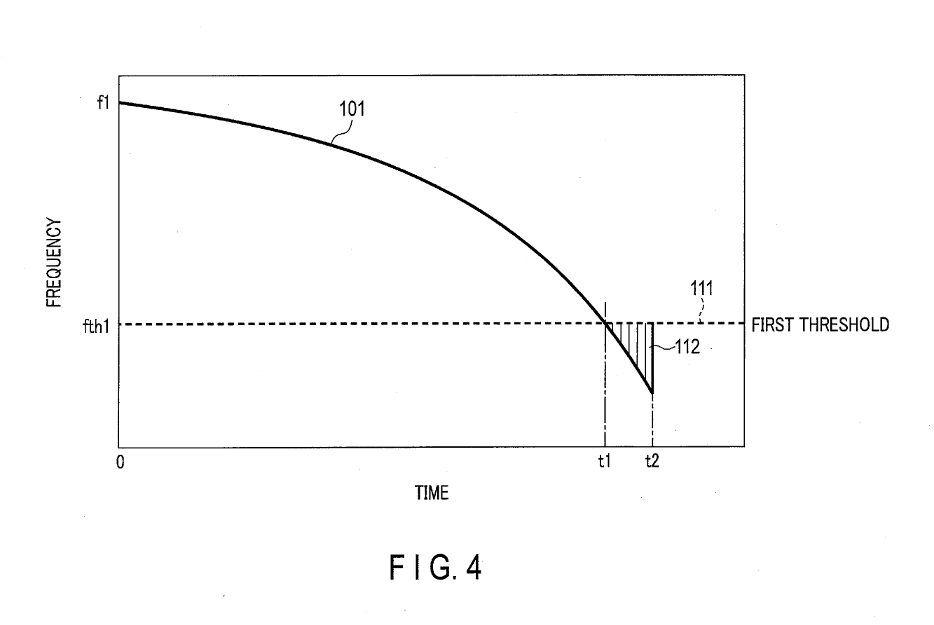

An operation relating to the stop or reduction of output according to the present embodiment will be described with reference to a schematic view illustrated in FIG. 4. FIG. 4 schematically illustrates a change of a resonance frequency of the vibration system including the ultrasonic transducer 13 with respect to an elapsed time from an output start. At the time of treatment, the temperature of the first grasping piece 15 gradually increases. Along with such a temperature rise, the resonance frequency of the vibration system including the first grasping piece 15 decreases. In the present embodiment, the processor 40 stops or reduces the output or notifies the user of the fact that the resonance frequency is decreasing (the temperature is rising) based on the decrease in the resonance frequency.

For example, the resonance frequency which was at a frequency f1 at a treatment start decreases with a lapse of time as indicated by a solid line 101 in FIG. 4. The processor 40 sets a first threshold at a frequency fth1 as indicated by a broken line 111, for example. The processor 40 calculates an integrated value 112 by integrating a difference between the resonance frequency and the first threshold from a time (time t1 in FIG. 4) when the resonance frequency of the vibration system has reached the first threshold. When the integrated value 112 exceeds a predetermined second threshold (time t2 in FIG. 4), the processor 40 stops or reduces the output or notifies that the integrated value 112 has reached the second threshold.

When a tissue is present between the first grasping piece 15 and the second grasping piece 16 and the tissue is being cut, heat generated in the first grasping piece 15 is used for the cutting of the tissue. Therefore, when there is a tissue between the first grasping piece 15 and the second grasping piece 16 before the tissue is divided, a temperature rises gently as compared with a case where there is no tissue between the first grasping piece 15 and the second grasping piece 16. Therefore, an increase rate of the integrated value of the difference between the resonance frequency of the vibration system and the first threshold is relatively small before the tissue is divided, and the increase rate of the integrated value of the difference between the resonance frequency of the vibration system and the first threshold is relatively large after the tissue has been divided. Therefore, it is possible to detect the division of the tissue by using the integrated value of the difference between the resonance frequency of the vibration system and the first threshold.

In general, it is preferable to set a resonance frequency corresponding to a temperature at which a tissue is divided as a value of the first threshold. An example of the temperature at which the tissue is divided is generally 200° C. or the like.

The frequency f1 of the vibration system at the start of treatment and the frequency fth1 of the first threshold are within a range of about 40 kHz to 60 kHz, for example. As an example, when the resonance frequency of the vibration system including the ultrasonic transducer 13 and the rod member 14 is 46 kHz to 48 kHz as described above, the frequency f1 at the start of treatment and the frequency fth1 of the first threshold also become values in the range of 46 kHz to 48 kHz. The time t1 at which the resonance frequency of the vibration system reaches the first threshold and the time t2 at which the integrated value reaches the second threshold are values within the range of several seconds to a dozen or so seconds, for example.

<Output Control Process>

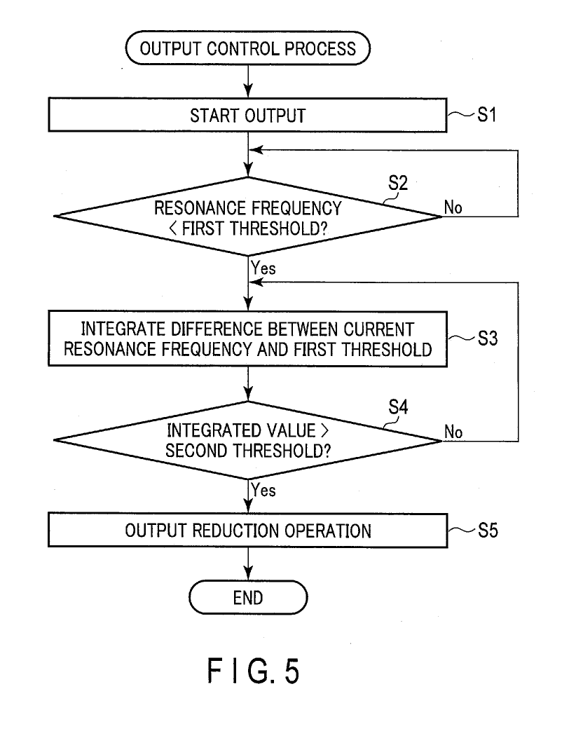

The control of the output by the processor 40 will be described with reference to the flowchart illustrated in FIG. 5. An output control process illustrated in FIG. 5 starts, for example, when the operation button 19 is pushed by the user.

In Step S1, the output control unit 43 of the processor 40 causes the energy output source 50 to start outputting ultrasonic waves. The energy output source 50 supplies predetermined power to the ultrasonic transducer 13 under the control of the processor 40. As a result, the first grasping piece 15 vibrates at an ultrasonic frequency to perform treatment of a living tissue.

First, the processor 40 causes the energy output source 50 to perform a scan operation of an output frequency, and then, starts control using a phase locked loop (PLL) to cause the energy output source 50 to output power. That is, the output control unit 43 of the processor 40 causes the energy output source 50 to output power while gradually changing the output frequency from a high frequency to a low frequency, for example, in the range including the resonance frequency of the vibration system including the ultrasonic transducer 13. The resonance frequency acquisition unit 41 of the processor 40 acquires a current and a voltage at this time and acquires a frequency at which phases of the current and the voltage coincide with each other, that is, the resonance frequency of the vibration system. The processor 40 uses the acquired resonance frequency as an initial value and makes the output frequency follow the resonance frequency of the vibration system using the PLL thereafter. Subsequently, the PLL is used to vibrate the first grasping piece 15 at the resonance frequency. In general, the temperature of the first grasping piece 15 gradually increases so that the resonance frequency of the vibration system gradually decreases as illustrated in FIG. 4.

In Step S2, the processor 40 determines whether the current resonance frequency of the vibration system is lower than a predetermined first threshold. If the resonance frequency is not lower than the first threshold, the process repeats Step S2. That is, the processor 40 continues the output control using the PLL to continue the treatment using the ultrasonic treatment instrument 2. When the resonance frequency has become lower than the first threshold, the process proceeds to Step S3.

Although it is described herein that the resonance frequency is lower than the first threshold, the value of the resonance frequency used for comparison is not limited to an instantaneous value but may be a moving average value or the like in order to prevent erroneous determination due to noise or the like.

In Step S3, the integrated value calculation unit 42 of the processor 40 integrates a difference between the current resonance frequency of the vibration system and the first threshold to calculate an integrated value. In Step S4, the processor 40 determines whether the integrated value is larger than a predetermined second threshold. When the integrated value is not larger than the second threshold, the process returns to Step S3. That is, the integration of the difference between the resonance frequency and the first threshold is repeated. As a result, the integrated value gradually increases. When the integrated value has become larger than the second threshold, the process proceeds to Step S5.

In Step S5, the output control unit 43 of the processor 40 performs an output reduction operation. The output reduction operation is, for example, an operation of stopping the output of power from the energy output source 50. The output reduction operation may be, for example, an operation of decreasing the output power from the energy output source 50. The output reduction operation may be an operation of notifying the user that the integrated value has reached the second threshold using the notification unit 64. This operation may be, for example, display on a display, an output of a notification sound from a speaker, or the like. The output reduction operation may be a combination of the stop or decrease of the output power and the notification. The output control process is ended after the processing of Step S5.

The first threshold and the second threshold may be values set in advance. For example, the processor 40 reads and sets the first threshold and the second threshold stored in a storage medium 48 at the time of activation. The energy control device 3 may be configured such that the first threshold and the second threshold can be changed based on an instruction from the user input via an input unit 62.

The first threshold and the second threshold may be different for each model of the ultrasonic treatment instrument 2. Therefore, the first threshold and the second threshold may be set as follows. That is, for example, identification information indicating a model of the ultrasonic treatment instrument 2 is stored in the storage medium 25 of the ultrasonic treatment instrument 2. Further, a correspondence between the first threshold and the second threshold for each model is stored in the storage medium 48 of the energy control device 3. The processor 40 reads the identification information on the model from the storage medium 25 of the ultrasonic treatment instrument 2 and uses the first threshold and the second threshold corresponding to the identification information. Even if the model information of the ultrasonic treatment instrument 2 is not stored in the storage medium 25, for example, different resistors may be provided in the ultrasonic treatment instrument 2 depending on the model of the ultrasonic treatment instrument 2, and the energy control device 3 may acquire a resistance value of the resistor at the time of being connected to the ultrasonic treatment instrument 2 to discriminate the model of the ultrasonic treatment instrument 2. The first threshold and the second threshold may be stored in the storage medium 25 of the ultrasonic treatment instrument 2, and the processor 40 may read such information to set the first threshold and the second threshold.

The energy control device 3 may be configured such that a type of the output reduction operation or presence or absence of the output reduction operation according to the present embodiment is switched based on an instruction of the user input via the input unit 62.

Although the description has been given in the above example by exemplifying the case where the integrated value is the integrated value of the difference between the first threshold and the resonance frequency at the corresponding point in time, the invention is not limited thereto. The integrated value may be an integrated value of a difference between a predetermined frequency other than the first threshold and a resonance frequency at the corresponding point in time

The case where only one second threshold is set is illustrated in the above example, but two or more second thresholds may be set. For example, the energy control device 3 may be configured such that the output is reduced when the integrated value of the difference between the resonance frequency and the first threshold becomes larger than a first second threshold, and the output is stopped when the integrated value of the difference between the resonance frequency and the first threshold becomes larger than a second threshold.

In the present embodiment, the ultrasonic treatment instrument 2 has been described as the instrument that performs treatment by ultrasonic vibration of the first grasping piece 15, but is not limited thereto. The ultrasonic treatment instrument 2 may have a function as a high-frequency treatment instrument that applies a high-frequency voltage between the first grasping piece 15 and the second grasping piece 16 in addition to the ultrasonic vibration. The high-frequency treatment instrument causes a high-frequency current to flow to a tissue to be treated to perform treatment with heat generated by the current flowing through the tissue. The ultrasonic treatment instrument 2 may be a treatment instrument in which a heater is provided on the first grasping piece 15 or the second grasping piece 16 to treat a tissue to be treated by heat of the heater together with ultrasonic vibration.

According to the treatment system 1 according to the present embodiment, the ultrasonic vibration of the first grasping piece 15 is reduced or stopped when the treatment of the living tissue to be treated has been completed and the tissue has been divided. Therefore, the ultrasonic vibration of the first grasping piece 15 is prevented while the first grasping piece 15 and the pad member 21 of the second grasping piece 16 are in contact with each other. As a result, abrasion or deformation of the pad member 21 is suppressed.

Further, the determination on whether to reduce or stop the ultrasonic vibration of the first grasping piece 15 is made based on the fact that the integrated value of the difference between the resonance frequency and the first threshold has become larger than the second threshold. Here, a situation of a living tissue between the first grasping piece 15 and the second grasping piece 16 is reflected on the difference between the resonance frequency and the first threshold, that is, a degree of the decrease in the resonance frequency when the resonance frequency has become lower than the first threshold. Therefore, the appropriate output control according to the treatment situation can be realized.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An energy control device that supplies power to an ultrasonic treatment instrument comprising an end effector performing treatment using ultrasonic vibration generated by an ultrasonic transducer, the energy control device comprising:
an energy source configured to output the power to the ultrasonic transducer; and
at least one integrated circuit configured to:
acquire a resonance frequency of a vibration system including the ultrasonic transducer;
calculate an integrated value of a difference between the resonance frequency and a predetermined frequency after the acquired resonance frequency has become a first threshold; and
perform at least one of causing the energy source to stop or reduce an output of the power to the ultrasonic transducer and notifying that the integrated value has reached the second threshold when the integrated value has reached a second threshold.

2. The energy control device according to claim 1, wherein
the integrated circuit is configured to calculate an integrated value of a difference between the resonance frequency and the first threshold as the integrated value.

3. The energy control device according to claim 1, wherein
the integrated circuit is configured to use a frequency corresponding to a temperature at which an object to be treated is cut as the first threshold.

4. The energy control device according to claim 1, further comprising an input device that receives an instruction from a user,
wherein the integrated circuit is configured to adjust at least one of the first threshold and the second threshold based on an input to the input device.

5. The energy control device according to claim 1, further comprising an input device that receives an instruction from a user,
wherein the integrated circuit is configured to switch a type of an operation or presence or absence of an operation when the integrated value has reached the second threshold based on an input to the input device.

6. The energy control device according to claim 1, wherein
the predetermined frequency is the first threshold.

7. A treatment system comprising:
the energy control device according to claim 1;
the ultrasonic transducer configured to be supplied with power from the energy control device; and
the ultrasonic treatment instrument.

8. The treatment system according to claim 7, wherein
the end effector comprises a first grasping piece configured such that ultrasonic vibration is transmitted and a second grasping piece configured to be open and closed with respect to the first grasping piece, and
the second grasping piece comprises a pad member that is brought into contact with the first grasping piece in a state where the first grasping piece and the second grasping piece is closed.

9. The treatment system according to claim 7, wherein
the ultrasonic treatment instrument comprises a memory in which the first threshold and the second threshold are stored, and
the integrated circuit is configured to read the first threshold and the second threshold from the memory and use the read first and second thresholds when the ultrasonic treatment instrument is connected to the energy control device.

10. The treatment system according to claim 7, wherein
the ultrasonic treatment instrument comprises a first memory in which identification information is stored,
the energy control device comprises a second memory in which a correspondence between the identification information and the first threshold and a correspondence between the identification information and the second threshold are stored, and
the integrated circuit is configured to read the identification information from the first memory and to read the first threshold and the second threshold stored in the second memory corresponding to the identification information and use the read first and second thresholds when the ultrasonic treatment instrument is connected to the energy control device.
